SEQUENCE LISTING (6 sequences)

SEQ ID NO: 1 - DNA, 20 bp, Treponema pallidum
  source 1..20: mol_type = other DNA
tccgctacgactactacggt

SEQ ID NO: 2 - DNA, 20 bp, Treponema pallidum
  source 1..20: mol_type = other DNA
gagactctgatggatgctgc

SEQ ID NO: 3 - DNA, 20 bp, Treponema pallidum
  source 1..20: mol_type = other DNA
tcttctcaatgcatttcgac

SEQ ID NO: 4 - DNA, 20 bp, Treponema pallidum
  source 1..20: mol_type = other DNA
tctatagacgatttacaacc

SEQ ID NO: 5 - DNA, 20 bp, Treponema pallidum
  source 1..20: mol_type = other DNA
cacctatgcgctatacaaaa

SEQ ID NO: 6 - DNA, 20 bp, Treponema pallidum
  source 1..20: mol_type = other DNA
cgtggcattcaggaaaagga

(19) United States
(12) Patent Application Publication
(10) Pub. No.: US 2026/0160725 A1
(43) Pub. Date: Jun. 11, 2026

(54) SENSING PROBES AND ELECTROCHEMICAL SENSING PLATFORM FOR DETECTION OF SYPHILIS

(71) Applicants: The Penn State Research Foundation, University Park, PA (US); University of Maryland, Baltimore, Baltimore, MD (US); University of Maryland, Baltimore County, Baltimore, MD (US)

(72) Inventors: Dipanjan Pan, University Park, PA (US); Parikshit Moitra, University Park, PA (US); Ketan Dighe, University Park, PA (US); Maha Mohammad Shehadeh Alafeef, University Park, PA (US)

(73) Assignees: The Penn State Research Foundation, University Park, PA (US); University of Maryland, Baltimore, Baltimore, MD (US); University of Maryland, Baltimore County, Baltimore, MD (US)

(21) Appl. No.: 19/370,274

(22) Filed: Oct. 27, 2025

Related U.S. Application Data
(63) Continuation-in-part of application No. PCT/US2024/035608, filed on Jun. 26, 2024.
(60) Provisional application No. 63/510,202, filed on Jun. 26, 2023.
(60) Provisional application No. 63/712,572, filed on Oct. 28, 2024.

(51) Int. Cl. G01N 27/327; C12Q 1/689

(52) U.S. Cl. CPC G01N 27/3276; C12Q 1/689; G01N 27/3278

(57) ABSTRACT

Embodiments relate to apparatuses, methods, and systems for screening and detecting syphilis. In particular, embodiments relate to sensing probes designed to bind in complementary fashion to one or more target gene sequences associated with syphilis. Embodiments further relate to electrochemical sensors configured to detect the presence of one or more target gene sequences associated with syphilis. The target gene sequences may include Tp47, polA, and TprE. These targets have conserved and distinctive sequences, enabling reliable detection even in samples with low bacterial loads or genetic mutations that may affect other diagnostic approaches.

### CROSS-REFERENCE TO RELATED APPLICATIONS

This patent application is a continuation-in-part application of International Patent Application No. PCT/US2024/035608, filed on Jun. 26, 2024, which is related to and claims the benefit of priority of U.S. Provisional Application 63/510,202, filed on Jun. 26, 2023. This patent application is further related to an claims the benefit of priority of U.S. Provisional Application No. 63/712,572, filed on Oct. 28, 2024. The entire contents of these applications are incorporated by reference.

### STATEMENT REGARDING FEDERALLY SPONSORED RESEARCH DEVELOPMENT

This invention was made with government support under Contract No. 75D30122C15492 awarded by the Center for Disease Control/DHHS. The Government has certain rights in the invention.

### INCORPORATION BY REFERENCE STATEMENT REGARDING SEQUENCE LISTINGS

The content of the XML file of the ST.26 SEQUENCE LISTING named “0073605-001101.xml”, which is 6170 bytes in size, was created on Oct. 27, 2025, and is electronically submitted herewith via Patent Center, and is hereby incorporated by reference in its entirety.

### FIELD

Embodiments relate to apparatuses, methods, and systems for screening and detecting syphilis. In particular, embodiments relate to sensing probes designed to bind in complementary fashion to one or more target gene sequences associated with syphilis. Embodiments further relate to electrochemical sensors configured to detect the presence of one or more target gene sequences associated with syphilis.

### BACKGROUND

Syphilis is a multistage sexually transmitted infection caused by the bacterium Treponema pallidum subsp. pallidum. It remains a significant public health concern worldwide, with an increasing incidence reported in many industrialized countries over the past decade. The disease progresses through distinct stages—primary, secondary, latent, and tertiary—each presenting different clinical features and diagnostic challenges. Accurate and timely diagnosis across all stages is critical for effective treatment and prevention of transmission, including vertical transmission from mother to infant.

Current diagnostic methods for syphilis include serological tests, darkfield microscopy, and nucleic acid amplification techniques such as polymerase chain reaction (PCR). While serological assays are widely used, they often lack sensitivity during early stages, particularly in primary syphilis where chancres may be painless and resolve spontaneously. Darkfield microscopy can directly visualize T. pallidum but requires specialized equipment and skilled personnel, limiting its routine use. PCR offers a sensitive alternative for detecting T. pallidum DNA, especially in primary syphilis, but its application is constrained by the need for sophisticated laboratory infrastructure and expertise. Moreover, PCR-based tests currently available are not FDA-approved point-of-care (POC) options and are often limited in sensitivity for secondary and latent stages.

### SUMMARY

Despite numerous diagnostic approaches, there remains no single, universally accepted test that can reliably detect T. pallidum across all stages of syphilis. The development of rapid, accurate, and easy-to-use diagnostic tools, particularly at the point of care, is an ongoing clinical need. Such advancements would facilitate early detection, prompt treatment, and better control of syphilis transmission, especially in resource-limited settings and during the early stages of infection.

The present disclosure therefore relates to sensing probes including anti-sense oligonucleotides (ASOs) that specifically bind to target gene sequences associated with syphilis. These ASOs are engineered to hybridize in a complementary fashion to their target gene sequences. The ASOs are configured to target multiple genetic regions of T. pallidum, including Tp47, polA, and TprE. These target genes have been selected for their conserved and distinctive sequences, enabling reliable detection even in samples with low bacterial loads or genetic mutations that may affect other diagnostic approaches.

The present disclosure further relates to a molecular diagnostic platform for the rapid and sensitive detection of T. pallidum. Recognizing the urgent need for reliable POC diagnostic tools capable of directly detecting T. pallidum DNA across various stages of infection, the platform introduces an integrated electrochemical assay that circumvents the limitations of existing methods such as PCR and serology. The platform is designed to provide quick, accurate results with minimal sample processing, making it suitable for deployment in diverse clinical settings.

Embodiments leverage advanced nanomaterials and molecular recognition techniques to achieve high sensitivity and specificity without the need for nucleic acid amplification. For example, electrodes functionalized with the above described sensing probes may be employed.

Further, the platform can incorporate a sandwiched nanomaterial architecture utilizing graphene and plasmonic nanoparticles to enhance electrochemical signal transduction. This design allows for ultra-sensitive detection of T. pallidum DNA, with a rapid turnaround time (e.g., less than 10 minutes) from sample collection to result. The platform's minimal sample preparation, combined with its multiplexed detection capability, addresses critical gaps in current syphilis diagnostics, particularly for early-stage infections where existing tests often lack sufficient sensitivity.

Ultimately, the present disclosure aims to facilitate early, accurate, and accessible diagnosis of syphilis, ultimately improving patient outcomes and supporting public health efforts to control the spread of this multistage STI.

In an exemplary embodiment. an apparatus for detecting syphilis includes a first sensing probe having a sequence that is complementary to a first target gene sequence; a second sensing probe having a sequence that is complementary to a second target gene sequence in close proximity to the first target gene sequence; a third sensing probe having a sequence that is complementary to a third target gene sequence; and a fourth sensing probe having a sequence that is complementary to a fourth target gene sequence in close proximity to the third target gene sequence.

In some embodiments, the first and second target gene sequences and the third and fourth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the first and second sensing probes are differentially functionalized with plasmonic nanoparticles, and wherein the third and fourth sensing probes are differentially functionalized with plasmonic nanoparticles.

In some embodiments, wherein the apparatus further includes a fifth sensing probe having a sequence that is complementary to a fifth target gene sequence; and a sixth sensing probe having a sequence that is complementary to a sixth target gene sequence in close proximity to the fifth target gene sequence.

In some embodiments, the first and second target gene sequences, the third and fourth target gene sequences, and the fifth and sixth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the first and second sensing probes are differentially functionalized with plasmonic nanoparticles, wherein the third and fourth sensing probes are differentially functionalized with plasmonic nanoparticles, and wherein the third and fourth sensing probes are differentially functionalized with plasmonic nanoparticles.

In an exemplary embodiment, an apparatus for detecting syphilis includes an electrode; one or more electrode sensing probes immobilized on a surface of the electrode, wherein a first end of the one or more electrode sensing probes is immobilized to the surface of the electrode and a second end of the one or more electrode sensing probes is functionalized with a first plasmonic nanoparticle; and one or more solution sensing probes provided in a solution further including a patient sample and optionally a nucleic acid extraction buffer, wherein a first end of the one or more solution sensing probes is functionalized with a second plasmonic nanoparticle and a second end of the one or more solution sensing probes is functionalized with an electrochemical configured to produce a measurable signal.

In some embodiments, the one or more electrode sensing probes include a first electrode sensing probe having a sequence that is complementary to a first target gene sequence, and a second electrode sensing probe having a sequence that is complementary to a third target gene sequence; and wherein the one or more solution sensing probes include a first solution sensing probe having a sequence that is complementary to a second target gene sequence in close proximity to the first target gene sequence, and a second solution sensing probe having a sequence that is complementary to a fourth target gene sequence in close proximity to the third target gene sequence.

In some embodiments, the first and second target gene sequences and the third and fourth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the one or more electrode sensing probes include a first electrode sensing probe having a sequence that is complementary to a first target gene sequence, a second electrode sensing probe having a sequence that is complementary to a third target gene sequence, and a third electrode sensing probe having a sequence that is complementary to a fifth target gene sequence; and the one or more solution sensing probes include a first solution sensing probe having a sequence that is complementary to a second target gene sequence in close proximity to the first target gene sequence, a second solution sensing probe having a sequence that is complementary to a fourth target gene sequence in close proximity to the third target gene sequence, and a third solution sensing probe having a sequence that is complementary to a sixth target gene sequence in close proximity to the fifth target gene sequence.

In some embodiments, the first and second target gene sequences, the third and fourth target gene sequences, and the fifth and sixth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the electrochemical reporter is methylene blue.

In some embodiments, the electrode includes a linking compound covalently attached to the surface of the electrode and configured to facilitate immobilization of the one or more electrode sensing probes to the surface of the electrode.

In some embodiments, the linking compound is carboxylic acid.

In an exemplary embodiment, a method for detecting syphilis includes collecting a sample including nucleic acid from a subject; combining the sample with a solution including solution sensing probes and optionally a nucleic acid extraction buffer; providing an apparatus including an electrode, and one or more electrode sensing probes immobilized on a surface of the electrode, wherein a first end of the one or more electrode sensing probes is immobilized to the surface of the electrode and a second end of the one or more electrode sensing probes is functionalized with a first plasmonic nanoparticle, and one or more solution sensing probes provided in a solution further including a patient sample and optionally a nucleic acid extraction buffer, wherein a first end of the one or more solution sensing probes is functionalized with a second plasmonic nanoparticle and a second end of the one or more solution sensing probes is functionalized with an electrochemical configured to produce a measurable signal; and applying the solution to the apparatus.

In some embodiments, the one or more electrode sensing probes include a first electrode sensing probe having a sequence that is complementary to a first target gene sequence, and a second electrode sensing probe having a sequence that is complementary to a third target gene sequence; and the one or more solution sensing probes include a first solution sensing probe having a sequence that is complementary to a second target gene sequence in close proximity to the first target gene sequence, and a second solution sensing probe having a sequence that is complementary to a fourth target gene sequence in close proximity to the third target gene sequence.

In some embodiments, the first and second target gene sequences and the third and fourth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the one or more electrode sensing probes include a first electrode sensing probe having a sequence that is complementary to a first target gene sequence, a second electrode sensing probe having a sequence that is complementary to a third target gene sequence, and a third electrode sensing probe having a sequence that is complementary to a fifth target gene sequence; and the one or more solution sensing probes include a first solution sensing probe having a sequence that is complementary to a second target gene sequence in close proximity to the first target gene sequence, a second solution sensing probe having a sequence that is complementary to a fourth target gene sequence in close proximity to the third target gene sequence, and a third solution sensing probe having a sequence that is complementary to a sixth target gene sequence in close proximity to the fifth target gene sequence.

In some embodiments, the first and second target gene sequences, the third and fourth target gene sequences, and the fifth and sixth target gene sequences are different pairs selected from SEQ ID NO 1 and SEQ ID NO 2, SEQ ID NO 3 and SEQ ID NO 4, and SEQ ID NO 5 and SEQ ID NO 6.

In some embodiments, the electrochemical reporter is methylene blue.

Other details, objects, and advantages of our apparatuses for screening and detecting syphilis, methods for screening and detecting syphilis, and systems for screening and detecting syphilis will become apparent as the following description of certain exemplary embodiments thereof proceeds.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a table of target gene sequences correlating to syphilis.

FIG. 2 is a table of exemplary sensing probes with sequences complementary to the target gene sequences included in FIG. 1.

FIG. 3 is a graph showing target binding energies and binding site disruption energies of exemplary sensing probes.

FIG. 4 shows a schematic representation of an exemplary method for screening and detecting syphilis.

FIG. 5 shows a schematic representation of the chemistry behind an exemplary system for screening and detecting syphilis.

FIG. 6 is a block diagram illustrating an exemplary system for screening and detecting syphilis.

FIG. 7 is a graph showing comparative change in current for functionalized electrodes in response to complementary strands of Tp47, polA, and TprE.

FIG. 8 is a graph showing change in current for a TprE targeted sensor with increasing concentration of synthetic DNA from T. pallidum.

FIG. 9 is a graph showing a representative cyclic voltammetry (CV) curve obtained from a TprE targeted sensing probe functionalized electrochemical sensor in the presence of its target DNA hybridized with a second sensing probe pair, thus forming a detectable sensing probe pair.

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

Embodiments generally relate to apparatuses, methods, and systems designed for the accurate screening and detection of syphilis. These may include sensing probes configured to selectively identify a target gene sequence or a region thereof that correlates with syphilis.

The sensing probes may include anti-sense oligonucleotides (ASOs) designed to bind specifically to target gene sequences associated with syphilis. These ASOs are engineered to hybridize in a complementary fashion to their target gene sequences. For example, ASOs have nucleotide sequences that complement the nucleotide sequence of a target gene (e.g., adenine (A) in an ASO sequence may complement and bind to uracil (U) or thymine (T) in a target gene sequence, cytosine (C) in an ASO sequence may complement and bind to guanine (G) in a target gene sequence, thymine (T) or uracil (U) in an ASO sequence may complement and bind to adenine (A) in a target gene sequence, and guanine (G) in an ASO sequence may complement and bind to cytosine (C) in a target gene sequence). The terms “anti-sense oligonucleotides” and “sensing probes” may be used interchangeably herein.

To screen and detect syphilis, at least one target gene sequence associated with syphilis must be identified. These sequences serve as specific binding sites for the sensing probes.

In some embodiments, the target gene sequences or regions thereof have a GC content between 40-60%, preferably between 50-60%. High GC content may enhance duplex stability due to triple hydrogen bonding. Too low GC content may reduce binding affinity and melting temperature. It is contemplated that target sequence length (e.g., 18-25 nt) can be tuned inversely with GC content to achieve equivalent binding energies. For example, a 15-mer with 60% GC content can yield a AG similar to a 20-mer with 45% GC content. This principle enables targeting “suboptimal” or variable GC regions without sacrificing hybridization affinity.

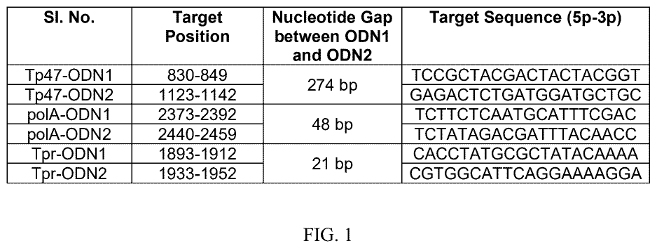

In some embodiments, target gene sequences or regions thereof that correlate with syphilis may be selected from the following sequences: SEQ ID NO 1 (TCCGCTACGACTACTACGGT), SEQ ID NO 2 (GAGACTCTGATGGATGCTGC), SEQ ID NO 3 (TCTTCTCAATGCATTTCGAC), SEQ ID NO 4 (TCTATAGACGATTTACAACC), SEQ ID NO 5 (CACCTATGCGCTATACAAAA), and SEQ ID NO 6 (CGTGGCATTCAGGAAAAGGA) (see FIG. 1). These sequences correspond to specific regions within genes of interest associated with syphilis.

SEQ ID NO 1 corresponds to a first region within the Tp47 gene, which encodes a lipoprotein antigen less prone to mutation and unaffected by known mutations such as A2058G. SEQ ID NO 2 pertains to a second, closely spaced region within the same Tp47 gene. Together, SEQ ID NO 1 and SEQ ID NO 2 represent two adjacent regions within the same gene.

Similarly, SEQ ID NO 3 corresponds to a first region within the DNA-directed DNA polymerase I gene, polA, which is also less susceptible to mutation. SEQ ID NO 4 relates to a nearby second region within the same gene.

SEQ ID NO 5 corresponds to a first region within the TprE gene, a treponemal-specific hypothetical protein, again chosen for its stability and resistance to known mutations. SEQ ID NO 6 relates to a second proximate region within TprE.

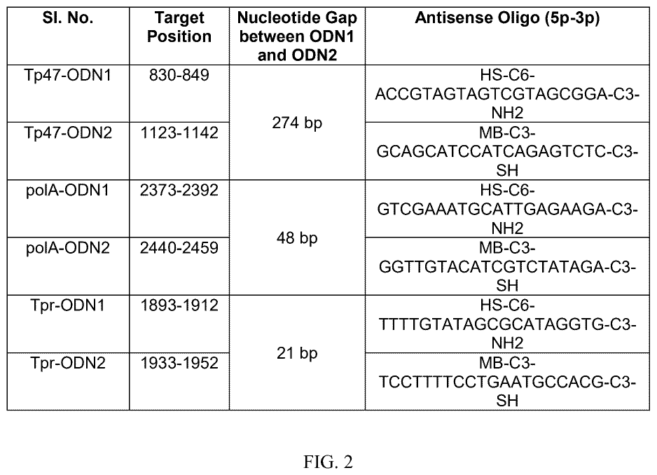

Sensing probes are designed to bind in a complementary fashion to these target sequences (see FIG. 2). For example, a sensing probe may have a sequence complementary to and configured to bind to SEQ ID NO 1, a sequence complementary to and configured to bind to SEQ ID NO 2, a sequence complementary to and configured to bind to SEQ ID NO 3, a sequence complementary to and configured to bind to SEQ ID NO 4, a sequence complementary to and configured to bind to SEQ ID NO 5, or a sequence complementary to and configured to bind to SEQ ID NO 6.

It is contemplated that the sensing probes may demonstrate a low self-complementarity, which may be important to prevent hairpin and dimer formation. Such secondary structures may drastically reduce hybridization efficiency and cause false-negative results.

It is further contemplated that preferred binding energies between the probes and sequences of interest may be approximately −7 to −9 kcal/mol, such as near −8 kcal/mol, thus balancing high affinity with selectivity. Beyond this range (e.g., ≤−12 kcal/mol), off-target risks may rise.

In exemplary embodiments, the sensing probes may be chosen in pairs and may be configured to bind to two closely spaced regions of a target gene sequence.

Any number of sensing probe pairs can be utilized. For example, a first sensing probe pair can be configured to complement and bind to two closely spaced regions of a first target gene sequence, a second sensing probe pair can be configured to complement and bind to two closely spaced regions of a second target gene sequence, a third sensing probe pair can be configured to complement and bind to two closely spaced regions of a third target gene sequence, a fourth sensing probe pair can be configured to complement and bind to two closely spaced regions of a fourth target gene sequence, a fifth sensing probe pair can be configured to complement and bind to two closely spaced regions of a fifth target gene sequence, etc.

For example, a first sensing probe pair can be configured to complement and bind to SEQ ID NO 1 and SEQ ID NO 2, a second sensing probe pair can be configured to complement and bind to SEQ ID NO 3 and SEQ ID NO 4, a third sensing probe pair can be configured to complement and bind to SEQ ID NO 5 and SEQ ID NO 6, etc.

The sensing probes may be functionalized or modified at either or both of their first end and their second end. The “first end” refers to a five prime end (5′ end) and the “second end” refers to a three prime end (3′ end).

In some embodiments, the sensing probes are functionalized at either their first end or their second end with a plasmonic (e.g., gold, silver, etc.) nanoparticle.

In some embodiments, each sensing probe of a pair may be differentially functionalized with detection probes (e.g., the first sensing probe functionalized with a detection probe at its first end and the second sensing probe functionalized with a detection probe at its second end) such that the ends functionalized with detection probes are in close proximity to each other when the sensing probes are bound to their respective regions of their target gene sequence. For example, a first sensing probe functionalized at its first end may be complementary to a first region of a target gene sequence, and a second sensing probe functionalized at its second end may be complementary to a second region of the target gene sequence that is in close proximity to the first region. As the detection probes are differentially functionalized, the detection probes may therefore be in close proximity to one another, thus influencing electron kinetics and/or enhancing detection sensitivity.

Embodiments further relate to electrochemical sensors configured to receive and analyze a sample to determine if the sample includes at least one target gene sequence correlating to syphilis. The sensor includes at least one sensing probe functionalized on an electrode. More specifically, at least one sensing probe may be immobilized on a surface of the electrode.

In some embodiments, the sensor can be used as a point-of-care (POC) test or point-of-site (POS) test, for example, as a rapid lab test, for screening and detecting syphilis.

In exemplary embodiments, the electrode is configured to detect an electrochemical signal (e.g., a redox signal, such as a current shift) in response to specific binding of sensing probes and corresponding target gene sequences present in a sample of a subject. In some embodiments, the electrode is in fluid contact with the sample after the sample is deposited onto the electrode.

The electrode may include any suitable electrode material. In some embodiments, the electrode can be a material selected from the group consisting of carbon, graphene, and graphene foam. Other embodiments can utilize other types of electrode materials.

To facilitate immobilization of sensing probes, the electrode surface can be modified to enable covalent attachment of the probes. In one embodiment, the surface may be carboxylated to accommodate the sensing probes.

In exemplary embodiments, the sensing probes may be chosen in pairs such that a first sensing probe is attached to the electrode (i.e., the electrode sensing probe) and the second sensing probe is introduced to a sample prior to the sample being deposited on the electrode (i.e., the solution sensing probe), as described in further detail below.

The electrode sensing probes may be attached to the electrode at one end (e.g., the first end of the second end) via a linking compound. The linking compound may be functionalized on a surface of the electrode and accommodate attachment of sensing probes to the electrode. For example, the linking compound may be an organic compound such as a carboxylic acid group that conjugates to an amino group of the electrode sensing probe.

The electrode sensing probes may be functionalized or modified at its opposite end with a first plasmonic nanoparticle.

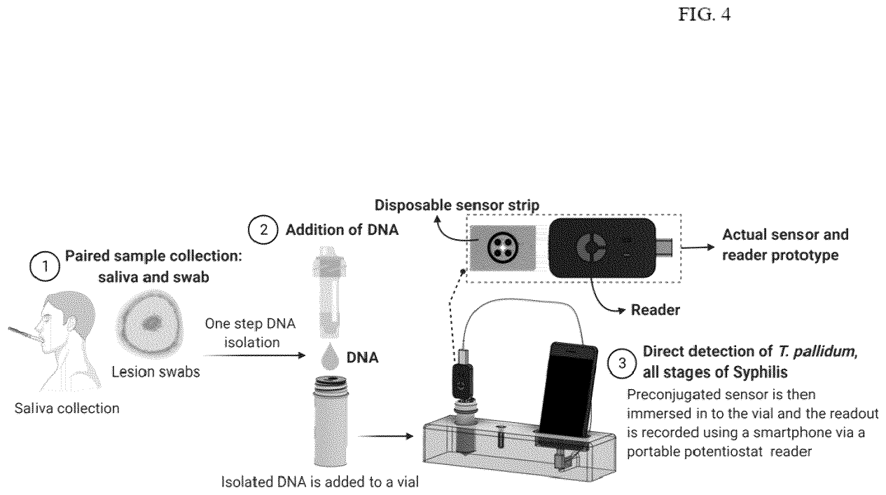

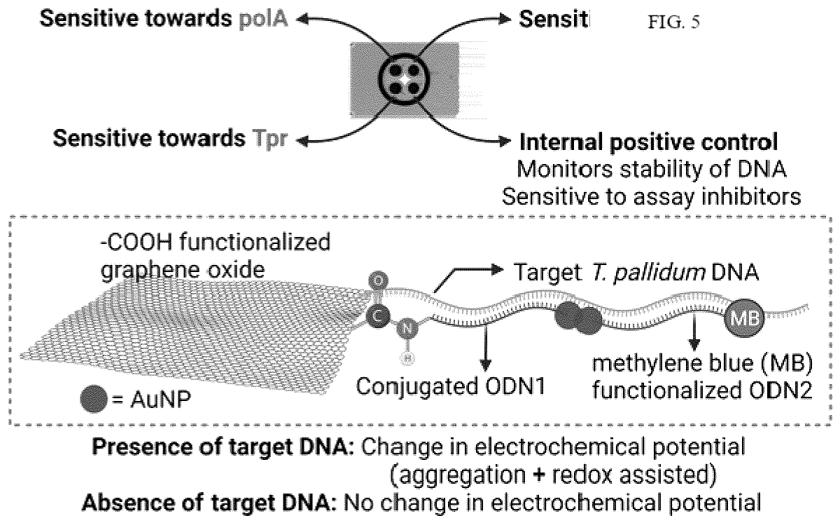

Referring to FIGS. 4 and 5, exemplary methods and systems for screening and detecting syphilis may include collecting a sample from a subject. It is contemplated that the sample may be collected using any suitable means, including but not limited to, an oral swab, a nasal swab, a cervical swab, a blood collecting swab, urine collection, or any other suitable means for collecting nucleic acid from the subject. In a preferred embodiment, the sample may be saliva, as it is non-invasive and can be collected easily.

It is further contemplated that the sample may be collected using any suitable instrument, including but not limited to, a cotton swab or any other suitable instrument for collecting nucleic acid from the subject.

The collected sample may then be introduced to a sensing solution to form an aqueous mixture.

In some embodiments, the sensing solution may include a nucleic acid extraction buffer configured to extract nucleic acids from the collected sample. In alternative embodiments, nucleic acids may not be extracted from the collected sample prior to application on the electrode. Extraction of nucleic acid and amplification of nucleic acid may be performed but are not requirements for using the electrochemical sensor, thus allowing sensing of a target gene sequence directly from the collected sample.

The solution sensing probes may also be introduced to the sample. In some embodiments, the solution sensing probes may form part of the sensing solution. In other embodiments, an additional solution including the solution sensing probes may be combined with the sample or sensing solution before the sample is deposited on the electrode.

The solution sensing probes may be functionalized or modified at one end (e.g., a first end or a second end) with a second plasmonic nanoparticle. The second plasmonic nanoparticle may be the same as or different than the first plasmonic nanoparticle.

As described above, the electrode sensing probes and the solution sensing probes may be differentially functionalized with detection probes such that the ends functionalized with detection probes are in close proximity to each other when the sensing probes are bound to their respective regions of their target gene sequence. For example, an electrode sensing probe functionalized at its first end may be complementary to a first region of a target gene sequence, and a solution sensing probe functionalized at its second end may be complementary to a second region of the target gene sequence that is in close proximity to the first region.

The solution sensing probes may be functionalized or modified at an opposite end with an electrochemical reporter, such that it may provide a measurable redox signal (e.g., current shift) by the electrode. In some embodiments, the electrochemical reporter may be methylene blue.

As can be appreciated from FIGS. 4 and 5, the aqueous mixture may then be deposited onto the surface of the electrochemical sensor (e.g., onto the electrode). As described above, the electrode sensing probes are functionalized on the electrode.

The electrode is configured to detect an electrochemical signal in response to specific binding of a sensing probe and a corresponding target gene sequence in the sample of a subject. In some embodiments, the electrochemical signal is a cyclic voltammetry (CV) signal, a differential pulse voltammetry (DPV) signal, or any other suitable signal. In some embodiments, the electrochemical signal, for example a redox current flowing through the electrode and/or an electrical impedance across the electrode, is in response to specific binding of a sensing probe and a corresponding target gene sequence in a sample of a subject. Moreover, as the sensing probes are differentially functionalized, the detection probes may therefore be in close proximity to one another, thus influencing electron kinetics and/or enhancing detection sensitivity.

Without wishing to be bound by theory, it is understood that only in the presence of a target gene sequence will a sensing probe-gene hybridized complex form leading to the agglomeration of plasmonic nanoparticles and restricting the electrochemical reporter near the electrode surface.

An electrode may include any number of electrode sensing probes. A sensor may include at least a first electrode sensing probe configured to complement and bind to a first target gene sequence, a second electrode sensing probe configured to complement and bind to a second target gene sequence, a third electrode sensing probe configured to complement and bind to a third target gene sequence, etc.

A solution may include any number of solution sensing probes. A solution may include at least a first solution sensing probe configured to complement and bind to a first target gene sequence, a second solution sensing probe configured to complement and bind to a second target gene sequence, a third solution sensing probe configured to complement and bind to a third target gene sequence, etc.

In some embodiments, the electrode is configured to detect the presence of a predetermined target gene sequence. For example, the system may be configured to detect one of Tp47, polA, and TprE.

In some embodiments, the sensor is configured to detect the presence of one or both of two predetermined target gene sequences. For example, the system may be configured to detect one or both of Tp47 and polA, one or both of polA and TprE, or one or both of Tp47 and TprE.

In some embodiments, the sensor is configured to detect the presence of one, two, or all of three predetermined target gene sequences. For example, the system may be configured to detect one, two, or all of Tp47, polA, and TprE.

It is an advantage to target three completely independent regions of a genome to provide unparalleled detection sensitivity and specificity covering multiple stages of syphilis. It can be anticipated that with this approach, the results may not be impacted by mutation and/or low level of T. pallidum in samples. Moreover, the changes of false-negative tests may be significantly less.

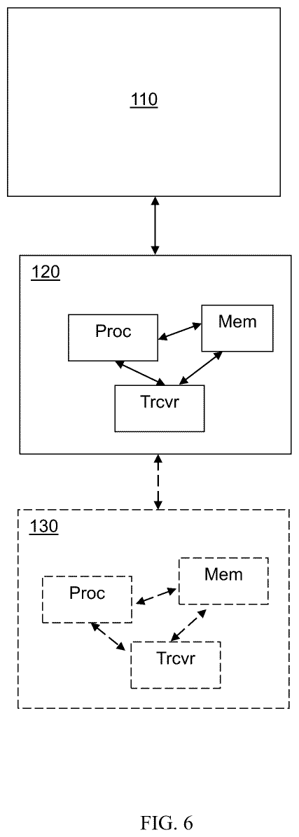

Referring to FIG. 6, the sensor 100 can be hardwire connected to an input/output device 110 (e.g., a smart phone, tablet, laptop computer, personal computer, computer device of a drone or robotic device, etc.), or the sensor 100 can be communicatively connected to the input/output device 110 via a network connection or wireless connection (e.g., internet connection, wide area network connection, near field communication connection, Bluetooth connection, etc.).

In some embodiments, the input/output device 110 can be configured to receive data from the sensor 100 for storage and analysis. In some implementations, the input/output device 110 can be configured as a server or cloud-based service providing device for storage and analysis of the data obtained via the sensor 100. The data can be communicated to a user via display device, which can be a tablet, smart phone, laptop computer, personal computer, or other type of terminal device. The display device can be effectuated via an application programming interface (API) and/or use of an application stored on the display device. It is contemplated that the input/output device 110 can include the display device, or the display device can be a separate device.

In some embodiments, the sensor 100 can alternatively or subsequently be sent to a central computer device 120 (e.g., a server, an operator workstation, etc.) that can be hardwire connected to the sensor 100 and/or the input/output device 110, or can be communicatively connected to the sensor 100 and/or the input/output 110 device via a network connection and/or a wireless connection. The central computer device 120 can be configured to store, analyze, and/or display data received from the sensor 100 and/or input/output device 110.

In some embodiments, the collected data can be continuously streamed to the input/output device 110 and/or the central computer device 120. In other embodiments, the collected data can be periodically streamed to the input/output device 110 and/or the central computer device 120 (e.g., non-continuously at pre-determined intervals). Embodiments of the sensor 100 can be configured to provide real time data collection.

The input/output device 110 and/or the central computer device 120 can be a computer device that can include a processor (Proc.) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr). The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of readings from the sensor 100 and/or control transmission of the collected data to input/output device 110 and/or the central computer device 120.

It should be noted that use of processors herein can include hardware, such as for example any one or combination of a Graphics Processing Unit (GPU), a Field Programmable Gate Array (FPGA), a Central Processing Unit (CPU), a microprocessor, a processor, etc. The processor can include one or more processing or operating modules. A processing or operating module can be a software or firmware operating module configured to implement any of the functions disclosed herein. The processing or operating module can be embodied as software and stored in non-transitory memory, the memory being operatively associated with the processor. A processing module can be embodied running a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, waveguides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or non-transitory machine-readable medium can be configured to store one or more instructions thereon. The instructions can be in the form of algorithms, program logic, etc. that cause the processor to execute any of the functions disclosed herein.

The processor can be in communication with other processors of other devices (e.g., additional external device, a computer system, a laptop computer, a desktop computer, etc.). An exemplary other device can be a Bluetooth enabled device, near field communication device, etc. Any of those other devices can include any of the exemplary processors disclosed herein as well as transceivers or other communication devices/circuitry to facilitate transmission and reception of wireless signals or other type of communicative connections.

Either the input/output device 110 and/or the central computer device 120 can be configured to be connected to other input devices and output devices. Examples of input devices can include a scanner device (e.g., scanner), a microphone, a keyboard, a touch screen, a button, a sensor a detector, or other type of input device. Examples of output devices can include a display, a printer, a speaker, or other type of output device.

As noted above, once collected data is transmitted to the input/output device 110 and/or the central computer device 120, the data can be analyzed and evaluated to determine whether a subject has syphilis. For example, electrochemical signals may be processed to determine if one or more target gene sequences are present in a sample collected from a subject. The signals may be processed to determine the presence or absence of target gene sequences in a samples.

### EXAMPLES

Six new probes were designed using a powerful computational algorithm to target three distinct regions of T. pallidum, i.e., two probes for each of (i) lipoprotein antigen Tp47; (ii) DNA-directed DNA polymerase I (polA) and (iii) treponemal conserved hypothetical protein (Tpr protein E) (FIGS. 1 and 2). While one of the probes was directly conjugated to the gold nanoparticles (5′-end) and connected to the carboxylated graphene surface (3′-end), the other probe was coupled with another gold nanoparticle (3′-end) and a redox reporter (e.g., methylene blue (MB)) at its 5′-end. We anticipated that only in presence of the target gene, the hybridized complex would form leading to the agglomeration of nanoparticles and restricting the redox reporter near the electrode surface (FIG. 4). Graphene, with high carrier mobility and large specific area, enables the detection of subtle changes on its surface which we utilized for the ultra-sensitive detection of syphilis. Thus, we presumed that the proposed sandwiched 3D/2D combination nanomaterial would significantly enhance the output signal (>50 fold) compared to either 3D or 2D, to detect the target gene without any nucleic acid amplification enabling PCR comparable sensitivity at the POC. Further, the advantage of targeting three completely independent regions of the genome was to provide unparallel detection sensitivity and specificity covering multiple stages of syphilis.

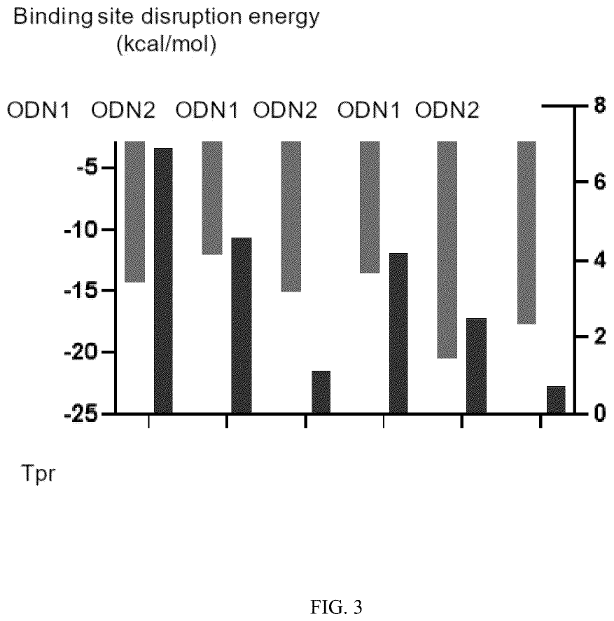

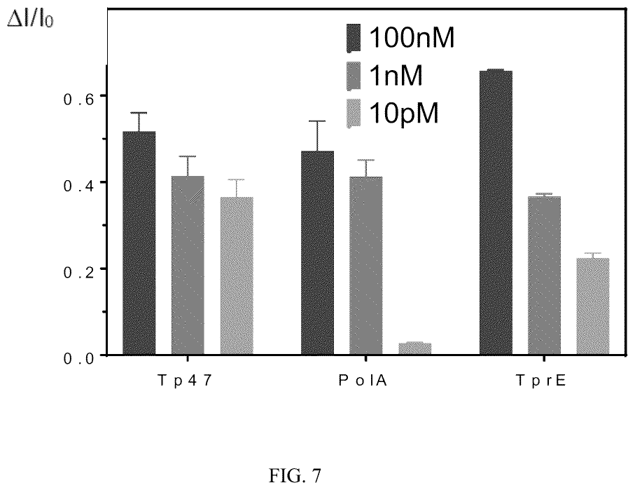

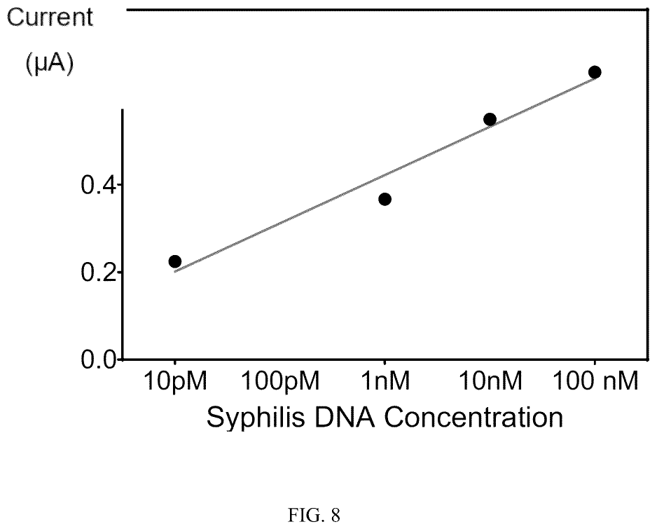

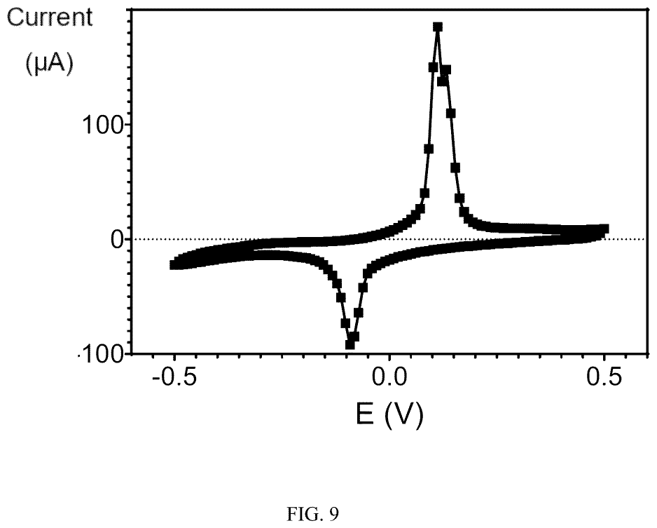

As we developed the probes targeted for different genetic segments of T. pallidum, we considered the formation of secondary structures of the probes in absence of their target DNA, comparative target binding and binding disruption energies (FIG. 3). We realized from prior experiences that absence of hairpin loop, more negative value of target binding energy and less positive value of binding disruption energy would generate a better targeting probe. It was found that among the three pairs of probes, Tpr targeting probes have the preferred condition followed by polA and Tp47. Further, the nucleotide gap between SEQ ID NO 1 and SEQ ID NO 2 is of 274 bp in case of Tp47, 48 bp for polA and 21 bp in case of Tpr (FIGS. 1 and 2). Hence, in presence of target DNA, the agglomeration of nanoparticles would be higher in case Tpr followed by polA and Tp47 (FIG. 4). Thus, we envisaged higher sensitivity and specificity from the Tpr targeted probes followed by polA and Tp47 targeted ones. Our strong preliminary electrochemical data further supported this theoretical understanding where the change in electrochemical potential is found to be the highest for Tpr, followed by polA and Tp47 (FIGS. 7-9).

Our preliminary results demonstrated that our platform has the capability to detect the three separate genetic segments of T. pallidum genome with an analytical limit of detection of ˜4.3 μM when tested against the synthetic T. pallidum DNA obtained from ATCC. The 3D Graphene Foam-based sensors exhibited enhanced electrochemical performance when compared to other carbon-based electrode materials. Each of the biosensor strip consisted of working electrode made up of 3D graphene foam (4 mm diameter), counter electrode made up of 3D graphene foam and reference electrode made up of screen-printed Ag/AgCl. The electrodes had polyimide substrate with silver connections. The thiol ends of both the probes were first used to cap citrate stabilized gold nanoparticles (AuNPs). The carboxylated graphene electrodes were covalently conjugated with 3′-amino group of a first probe. The change in the cyclic voltammetry (CV) curve of the sensor before and after the conjugation of the molecular probe confirmed the successful conjugation to the carboxylated graphene sensor surface. The extracted DNA sample was added to a vial containing the second probe with 5′-methylene blue (MB) and 3′-AuNP conjugation. The hybridized complex was then added onto the graphene surface containing the first probe. Using this screen-printed electrochemical-based platform we were successful in quantitatively measuring the genetic material of T. pallidum without any DNA extraction or amplification steps. A linear increase in current change was observed with increased addition of DNA concentration which is represented by the CV curve measured by the potentiostat reader device. Further, the functionalized sensors were found to be highly sensitive towards syphilis DNA compared to samples infected with Chlamydia trachomatis, Neisseria gonorrhea and HIV-1. The positive samples exhibited a high change in the current (FIGS. 7-9), whereas negligible change was observed in case of negative samples.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. An apparatus for detecting syphilis, the apparatus comprising:
a first sensing probe having a sequence that is complementary to a first target gene sequence;
a second sensing probe having a sequence that is complementary to a second target gene sequence in close proximity to the first target gene sequence;
a third sensing probe having a sequence that is complementary to a third target gene sequence; and
a fourth sensing probe having a sequence that is complementary to a fourth target gene sequence in close proximity to the third target gene sequence.

2. The apparatus of claim 1, wherein the first and second target gene sequences and the third and fourth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

3. The apparatus of claim 1, wherein the first and second sensing probes are differentially functionalized with plasmonic nanoparticles, and wherein the third and fourth sensing probes are differentially functionalized with plasmonic nanoparticles.

4. The apparatus of claim 1, further comprising:
a fifth sensing probe having a sequence that is complementary to a fifth target gene sequence; and
a sixth sensing probe having a sequence that is complementary to a sixth target gene sequence in close proximity to the fifth target gene sequence.

5. The apparatus of claim 4, wherein the first and second target gene sequences, the third and fourth target gene sequences, and the fifth and sixth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

6. The apparatus of claim 4, wherein the first and second sensing probes are differentially functionalized with plasmonic nanoparticles, wherein the third and fourth sensing probes are differentially functionalized with plasmonic nanoparticles, and wherein the third and fourth sensing probes are differentially functionalized with plasmonic nanoparticles.

7. An apparatus for detecting syphilis, the apparatus comprising:
an electrode;
one or more electrode sensing probes immobilized on a surface of the electrode, wherein a first end of the one or more electrode sensing probes is immobilized to the surface of the electrode and a second end of the one or more electrode sensing probes is functionalized with a first plasmonic nanoparticle; and
one or more solution sensing probes provided in a solution further including a patient sample and optionally a nucleic acid extraction buffer, wherein a first end of the one or more solution sensing probes is functionalized with a second plasmonic nanoparticle and a second end of the one or more solution sensing probes is functionalized with an electrochemical configured to produce a measurable signal.

8. The apparatus of claim 7, wherein the one or more electrode sensing probes comprise a first electrode sensing probe having a sequence that is complementary to a first target gene sequence, and a second electrode sensing probe having a sequence that is complementary to a third target gene sequence; and
wherein the one or more solution sensing probes comprise a first solution sensing probe having a sequence that is complementary to a second target gene sequence in close proximity to the first target gene sequence, and a second solution sensing probe having a sequence that is complementary to a fourth target gene sequence in close proximity to the third target gene sequence.

9. The apparatus of claim 8, wherein the first and second target gene sequences and the third and fourth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

10. The apparatus of claim 7, wherein the one or more electrode sensing probes comprise a first electrode sensing probe having a sequence that is complementary to a first target gene sequence, a second electrode sensing probe having a sequence that is complementary to a third target gene sequence, and a third electrode sensing probe having a sequence that is complementary to a fifth target gene sequence; and
wherein the one or more solution sensing probes comprise a first solution sensing probe having a sequence that is complementary to a second target gene sequence in close proximity to the first target gene sequence, a second solution sensing probe having a sequence that is complementary to a fourth target gene sequence in close proximity to the third target gene sequence, and a third solution sensing probe having a sequence that is complementary to a sixth target gene sequence in close proximity to the fifth target gene sequence.

11. The apparatus of claim 10, wherein the first and second target gene sequences, the third and fourth target gene sequences, and the fifth and sixth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

12. The apparatus of claim 7, wherein the electrochemical reporter is methylene blue.

13. The apparatus of claim 7, wherein the electrode includes a linking compound covalently attached to the surface of the electrode and configured to facilitate immobilization of the one or more electrode sensing probes to the surface of the electrode.

14. The apparatus of claim 13, wherein the linking compound is carboxylic acid.

15. A method for detecting syphilis, the method comprising:
collecting a sample comprising nucleic acid from a subject;
combining the sample with a solution comprising solution sensing probes and optionally a nucleic acid extraction buffer;
providing an apparatus comprising:
an electrode, and
one or more electrode sensing probes immobilized on a surface of the electrode, wherein a first end of the one or more electrode sensing probes is immobilized to the surface of the electrode and a second end of the one or more electrode sensing probes is functionalized with a first plasmonic nanoparticle, and
one or more solution sensing probes provided in a solution further including a patient sample and optionally a nucleic acid extraction buffer, wherein a first end of the one or more solution sensing probes is functionalized with a second plasmonic nanoparticle and a second end of the one or more solution sensing probes is functionalized with an electrochemical configured to produce a measurable signal; and
applying the solution to the apparatus.

16. The method of claim 15, wherein the one or more electrode sensing probes comprise a first electrode sensing probe having a sequence that is complementary to a first target gene sequence, and a second electrode sensing probe having a sequence that is complementary to a third target gene sequence; and
wherein the one or more solution sensing probes comprise a first solution sensing probe having a sequence that is complementary to a second target gene sequence in close proximity to the first target gene sequence, and a second solution sensing probe having a sequence that is complementary to a fourth target gene sequence in close proximity to the third target gene sequence.

17. The method of claim 16, wherein the first and second target gene sequences and the third and fourth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

18. The method of claim 15, wherein the one or more electrode sensing probes comprise a first electrode sensing probe having a sequence that is complementary to a first target gene sequence, a second electrode sensing probe having a sequence that is complementary to a third target gene sequence, and a third electrode sensing probe having a sequence that is complementary to a fifth target gene sequence; and
wherein the one or more solution sensing probes comprise a first solution sensing probe having a sequence that is complementary to a second target gene sequence in close proximity to the first target gene sequence, a second solution sensing probe having a sequence that is complementary to a fourth target gene sequence in close proximity to the third target gene sequence, and a third solution sensing probe having a sequence that is complementary to a sixth target gene sequence in close proximity to the fifth target gene sequence.

19. The method of claim 18, wherein the first and second target gene sequences, the third and fourth target gene sequences, and the fifth and sixth target gene sequences are different pairs selected from:
SEQ ID NO 1 and SEQ ID NO 2,
SEQ ID NO 3 and SEQ ID NO 4, and
SEQ ID NO 5 and SEQ ID NO 6.

20. The method of claim 15, wherein the electrochemical reporter is methylene blue.
